(19) United States
(12) Patent Application Publication
(10) Pub. No.: US 2013/0165813 A1
(43) Pub. Date: Jun. 27, 2013

(54) SENSOR FOR ACQUIRING MUSCLE PARAMETERS

(71) Applicants: Cheng-Hung Chang, Taichung City (TW); Kuan-Jen Fang, Tainan City (TW); Chun-Hsiang Huang, Tainan City (TW); Chueh-Shan Liu, Tainan City (TW); Yii-Tay Chiou, Kaohsiung City (TW); Yung-Ching Huang, Taipei City (TW)

(72) Inventors: Cheng-Hung Chang, Taichung City (TW); Kuan-Jen Fang, Tainan City (TW); Chun-Hsiang Huang, Tainan City (TW); Chueh-Shan Liu, Tainan City (TW); Yii-Tay Chiou, Kaohsiung City (TW); Yung-Ching Huang, Taipei City (TW)

(73) Assignee: INDUSTRIAL TECHNOLOGY RESEARCH INSTITUTE, HSINCHU (TW)

(21) Appl. No.: 13/448,297

(22) Filed: Apr. 16, 2012

(30) Foreign Application Priority Data
Dec. 23, 2011 (TW) 100148218

(51) Int. Cl. A61B 5/0488; A61B 5/11

(52) U.S. Cl. 600/546

(57) ABSTRACT

A sensor for acquiring EMG and MMG signals is provided, including a substrate, an inertial sensing element received in a hole of the substrate, a circuit element disposed on the substrate, a plurality of electrical connecting members connecting the inertial sensing element with the substrate, and a sensing ring disposed on the substrate and surrounding the hole. The electrical connecting members are flexible, and the circuit element and the sensing ring are disposed on opposite sides of the substrate.

### CROSS REFERENCE TO RELATED APPLICATIONS

This Application claims priority of Taiwan Patent Application No. 100148218, filed on Dec. 23, 2011, the entirety of which is incorporated by reference herein.

### BACKGROUND

1. Technical Field

The present disclosure relates to a sensor for acquiring muscle parameters, and in particular relates to a sensor for synchronously acquiring EMG and MMG signals.

2. Description of the Related Art

Please refer to FIG. 1, wherein two different sensors E and M are usually used for synchronously acquiring electromyography (EMG) and mechanomyography (MMG) signals. In general, the sensor E has electrodes and can be attached to skin for acquiring EMG signals. The other sensor M has inertial sensing elements or vibrarion/pressure sensing elements for tracking the motion of muscles or the skeleton and acquiring MMG signals.

Since some conventional sensors E and M are usually disposed apart from each other, providing an integrated micro sensor for synchronously acquiring EMG and MMG signals has become an important issue.

### SUMMARY

The disclosure provides a sensor for acquiring EMG and MMG signals including a substrate, an inertial sensing element received in a hole of the substrate, a circuit element disposed on the substrate, a plurality of electrical connecting members connecting the inertial sensing element with the substrate, and a sensing ring disposed on the substrate and surrounding the hole. The electrical connecting members are flexible, and the circuit element and the sensing ring are disposed on opposite sides of the substrate. Detailed description is given in the following embodiments with reference to the accompanying drawings.

### BRIEF DESCRIPTION OF THE DRAWINGS

The present disclosure can be more fully understood by reading the subsequent detailed description and examples with references made to the accompanying drawings, wherein:

FIG. 1 is a perspective diagram of two conventional sensors for respectively acquiring EMG and MMG signals;

FIG. 2A is a bottom view of a sensor for acquiring muscle parameters according to an embodiment of the disclosure;

FIG. 2B is a sectional view along A1-A1 of FIG. 2A;

FIG. 3 is a sectional view along A2-A2 of FIG. 2A;

FIG. 4 is a schematic view of a sensor attached to the surface of a human body for acquiring muscle parameters according to an embodiment of the disclosure; and

FIG. 5 is a schematic view of a sensor for acquiring muscle parameters having a protection layer disposed on the bottom side of the sensing ring and the inertial sensing element.

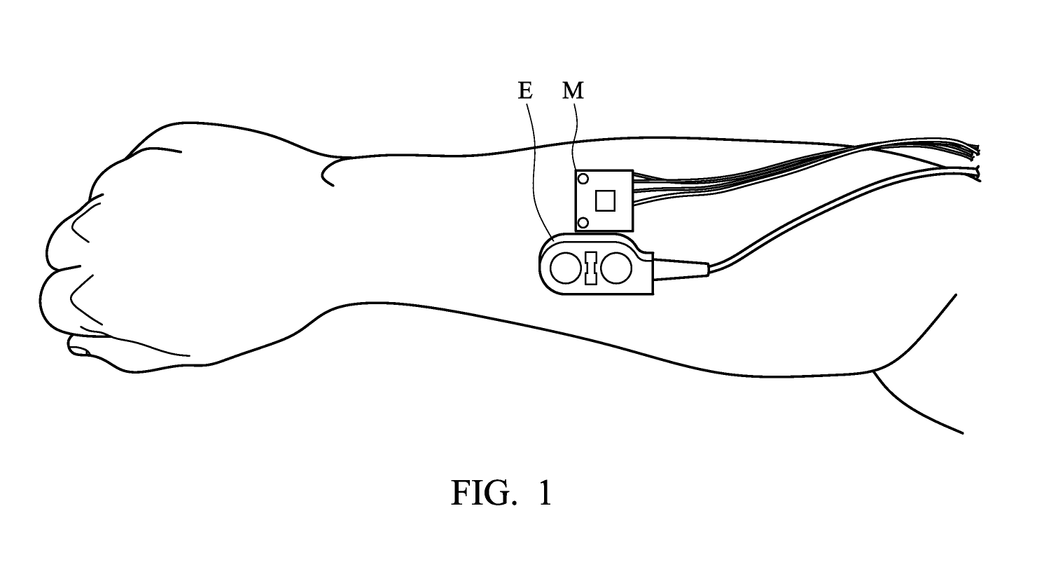

### DETAILED DESCRIPTION

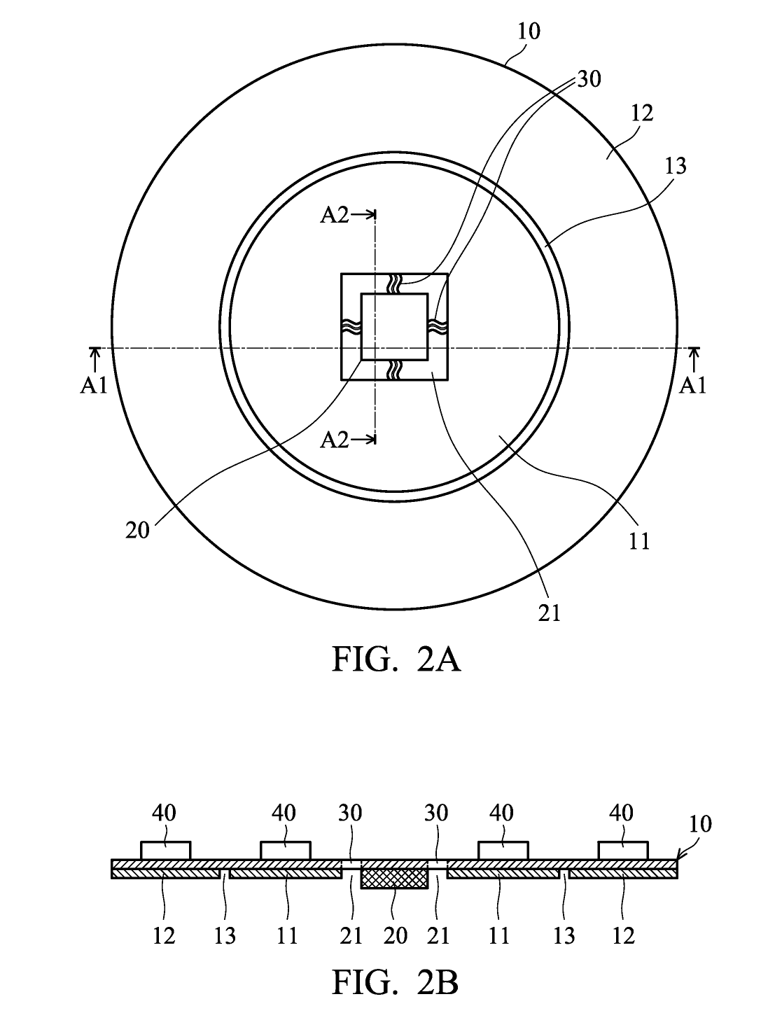

Referring to FIG. 2A, 2B, an embodiment of a sensor for acquiring muscle parameters is provided for synchronously acquiring electrophysiological and inertial signals, such as electromyography (EMG) and mechanomyography (MMG) signals. The sensor comprises a circular substrate 10, a rectangular inertial sensing element 20, a plurality of strip-shaped electrical connecting members 30, and at least one circuit element 40. As shown in FIG. 2A and 2B, the substrate 10 may be a flexible printed circuit (FPC) forming a rectangular or other shaped opening 21 at the center thereof for receiving the inertial sensing element 20. Additionally, four S-shaped, linear or curved electrical connecting members 30 are extended outward from four sides of the inertial sensing element 20 to the inner of the substrate 10, respectively, wherein the inertial sensing element 20 can be electrically connected to the circuit element 40 on the upper surface of the substrate 10 by the electrical connecting members 30.

Specifically, each of the electrical connecting members 30 in this embodiment is flexible and forms a suspension structure between the substrate 10 and the inertial sensing element 20. When the substrate 10 is pressed, the suspension structure may prevent the inertial sensing element 20 from interference caused by deformation of the substrate 10. In some embodiments, the electrical connecting members 30 may have polyimide (PI), and the electrical connecting members 30 and the substrate 10 may be integrally formed in one piece as an FPC, thus reducing the complexity of a mechanism and saving production cost.

As shown in FIGS. 2A and 2B, two sensing rings 11 and 12 are disposed on the lower surface of the substrate 10 with a recess 13 formed therebetween. It is noted that the sensing rings 11 and 12 on the lower surface of substrate 10 have capacitive sensing electrodes therein for acquiring the electromyography signals. Additionally, the sensor can also synchronously acquire the mechanomyography signals of a human body through the inertial sensing element 20, which is at the center of the substrate 10. The inertial sensing element 20 may comprise an inertial element (such as accelerometer gyroscope) or a vibrating element (such as manometer, microphone) for acquiring the mechanomyography signal of a human body. As the inertial sensing element 20 and the sensor ring 11 and 12 are respectively disposed at the center and on the lower surface of the substrate 10, they do not have to be disposed separately, so as to achieve miniaturization of the sensor. Hence, high resolution and accurate measurement of a small area of a human muscle can be achieved by the sensor.

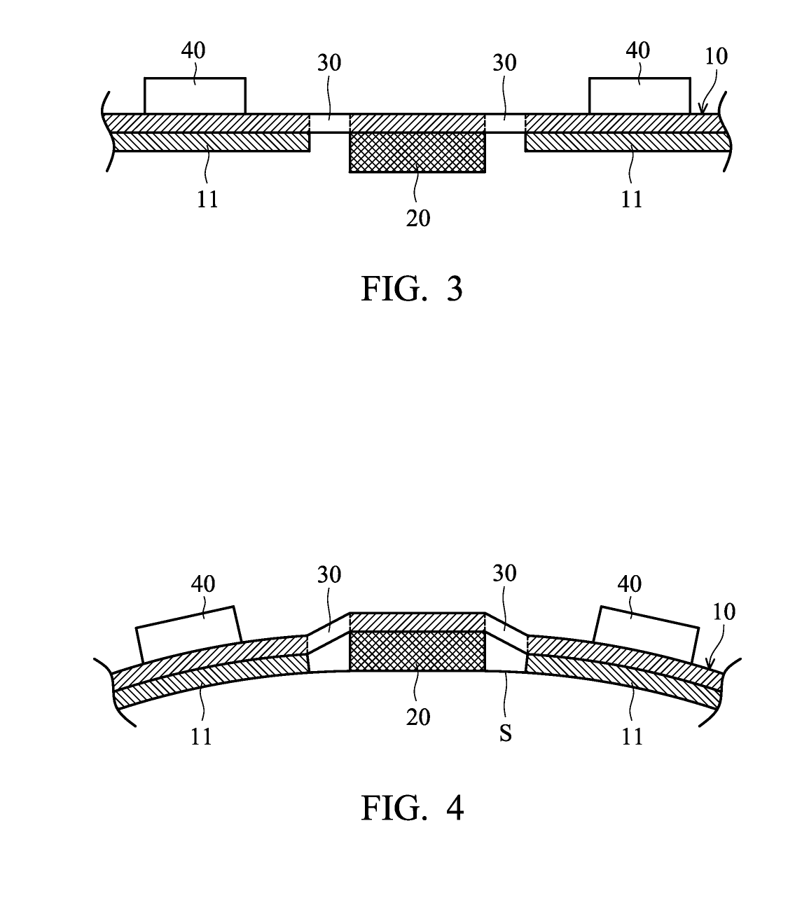

Referring to FIGS. 3 and 4, as the thickness of inertial sensing element 20 exceeds that of the sensing ring 11, the inertial sensing element 20 in the center of the substrate 10 is lower than the sensing ring 11 before the sensor is used (FIG. 3). When to the sensor is used to synchronously acquire the electromyography and mechanomyography signals, the inertial sensing element 20 and the sensing ring 11 can be attached to the human skin S, as shown in FIG. 4. Meanwhile, the inertial sensing element 20 and the electrical connecting members 30 are extruded upwardly by the human skin S. Since the electrical connecting members 30 are flexible, the inertial sensing element 20 and the circuit element 40 can remain electrically connected. Additionally, since the inertial sensing element 20 is suspended relative to the substrate 10 when the sensor is active, the inertial sensing element 20 can be protected from interference due to deformation of the substrate 10 and the sensing ring 11, thus improving the resolution and the sensitivity of the sensor.

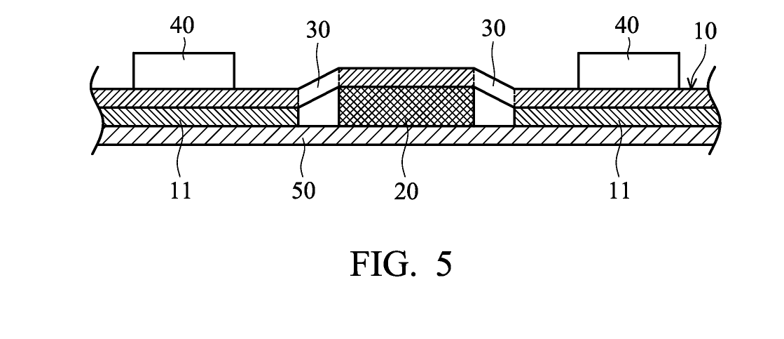

Referring to FIG. 5, another embodiment of a sensor further comprises a protection layer 50 disposed on the bottom side of the sensing ring 11 and the inertial sensing element 20. In this embodiment, the protection layer 50 may be made of flexible material, such as silicon gel or polyimide. The protection layer 50 not only covers and protects the sensing ring 11 and the inertial sensing element 20, but also increases the structural strength of the sensor, so as to prevent damage of the electrical connecting members 30 during usage.

In some embodiments, the protection layer 50 can also be disposed on the top side of the sensing ring 11 and the inertial sensing element 20, rather than the bottom side of the sensing ring 11 and the inertial sensing element 20. Additionally, the protection layer 50 can also encompass the whole sensor as a package structure (including bottom, top, and lateral sides), thus facilitating comprehensive protection of the sensor.

The disclosure provides a sensor for acquiring an electrophysiological signal and an inertial signal, such as EMG and MMG signals, or electrocardiography and respiratory physiological signals. The sensor comprises a substrate, an inertial sensing element, a circuit element, a plurality of electrical connecting members, and a sensing ring. The inertial sensing element is disposed in an opening of the substrate, the circuit element is disposed on the substrate, and the electrical connecting members are flexible and connect the inertial sensing element with the substrate. The inertial sensing element and the circuit element are electrically connected to each other through the electrical connecting members. The sensing ring is disposed on the substrate and surrounds the opening, and the circuit element and the sensing ring are disposed on opposite sides of the substrate.

Specifically, as the inertial sensing element and the sensor ring are located respectively at the center and on the lower surface of the substrate, they do not have to be disposed separately, so as to achieve miniaturization of the sensor. Hence, high resolution and accurate measurement in a small area of a human muscle can be achieved by the sensor. Moreover, since each of the electrical connecting members is flexible and forms a suspension structure between the substrate and the inertial sensing element, the inertial sensing element can be prevented from disturbance caused by deformation of the substrate when the substrate is pressed, so as to facilitate high sensitivity of measurement.

While the disclosure has been described by way of example and in terms of the preferred embodiments, it is to be understood that the disclosure is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A sensor for acquiring EMG and MMG signals, comprising:
a substrate, having an opening;
an inertial sensing element, disposed in the opening;
a circuit element, disposed on the substrate;
a plurality of electrical connecting members, connecting the inertial sensing element with the substrate, wherein the electrical members are flexible, and the circuit element and the circuit element are electrically connected to each other through the electrical connecting members; and
a sensing ring, disposed on the substrate and surrounding the opening, wherein the circuit element and the sensing ring are disposed on opposite sides of the substrate.

2. The sensor for acquiring EMG and MMG signals as claimed in claim 1, wherein the substrate is a flexible printed circuit, and the electrical members and the substrate are integrally formed in one piece.

3. The sensor for acquiring EMG and MMG signals as claimed in claim 1, wherein the inertial sensing element comprises an inertial element or a vibrating element for acquiring the MMG signal.

4. The sensor for acquiring EMG and MMG signals as claimed in claim 1, wherein the sensing ring comprises a capacitive sensing electrode for acquiring the EMG signal.

5. The sensor for acquiring EMG and MMG signals as claimed in claim 1, wherein the inertial sensing element has a rectangular structure, and the electrical connecting members are respectively extended from four sides of the inertial sensing element to the substrate.

6. The sensor for acquiring EMG and MMG signals as claimed in claim 1, wherein the electrical connecting members comprises polyimide.

7. The sensor for acquiring EMG and MMG signals as claimed in claim 1, wherein the electrical connecting members have an S-shaped structure.

8. The sensor for acquiring EMG and MMG signals as claimed in claim 1, wherein the sensor further comprises two sensing rings with a recess formed therebetween.

9. The sensor for acquiring EMG and MMG signals as claimed in claim 1, wherein the sensor further comprises a flexible protection layer covering the sensing ring and the inertial sensing element.

10. The sensor for acquiring EMG and MMG signals as claimed in claim 9, wherein the protection layer comprises silicon gel or polyimide.
